(19) United States
(12) Patent Application Publication
(10) Pub. No.: US 2007/0251026 A1
(43) Pub. Date: Nov. 1, 2007

(54) Unsaturated fatty substances for protecting the color of artificially dyed keratin fibers with respect to washing; and dyeing processes

(76) Inventors: Boris Lalleman, Paris (FR); Franck Giroud, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/783,770

(22) Filed: Apr. 12, 2007

Related U.S. Application Data
(60) Provisional application No. 60/792,955, filed on Apr. 19, 2006.

(30) Foreign Application Priority Data
Apr. 12, 2006 (FR) 0651335

(51) Int. Cl. A61K 8/00

(52) U.S. Cl. 8/405

(57) ABSTRACT

Disclosed herein is a process involving the use of at least one unsaturated fatty substance of formula (I) as defined herein as an agent for protecting color with respect to the washing of artificially dyed keratin fibers such as artificially dyed hair. Also disclosed herein is a process for protecting the color of artificially dyed keratin fibers with respect to washing, comprising applying to the fibers, before or after dyeing, at least one composition comprising, in a cosmetically acceptable medium, at least one unsaturated fatty substance of formula (I). Further disclosed herein is a process for dyeing keratin fibers, comprising applying to the keratin fibers a direct or oxidation dye composition (A) for a time that is sufficient to develop the color, and following or preceding this application, applying a composition (B) comprising, in a cosmetically acceptable medium, at least one unsaturated fatty substance of formula (I).

This application claims benefit of U.S. Provisional Application No. 60/792,955, filed Apr. 19, 2006, the contents of which are incorporated herein by reference. This application also claims benefit of priority under 35 U.S.C. § 119 to French Patent Application No. FR 06/51335, filed Apr. 12, 2006, the contents of which are also incorporated herein by reference.

Disclosed herein are unsaturated fatty substances of formula (I) as defined herein which can be used as agents for protecting color with respect to the washing of artificially dyed keratin fibers, for example, human keratin fibers such as the hair.

It is known practice to dye keratin fibers, for example, human keratin fibers such as the hair, with dye compositions comprising oxidation dye precursors, which are generally known as oxidation bases. These oxidation bases are colorless or weakly colored compounds, which, when combined with oxidizing products, give rise to colored compounds via a process of oxidative condensation. It is also known that the shades obtained, with these oxidation bases can be varied by combining them with couplers or coloration modifiers. The variety of molecules used as oxidation bases and couplers can allow a wide range of colors to be obtained.

It is also known practice to dye keratin fibers by direct dyeing. The process conventionally used in direct dyeing comprises applying to the keratin fibers direct dyes, which are colored and coloring molecules that have affinity for the fibers, leaving them to act, and then rinsing the fibers.

The colorations resulting therefrom may be particularly chromatic colorations, but are, however, temporary or semi-permanent since the nature of the interactions that bind the direct dyes to the keratin fiber and their desorption from the surface and/or core of the fiber are responsible for their weak dyeing power and their poor wash-fastness.

The artificial color of hair provided by a direct or oxidation dyeing treatment gradually attenuates as a result of repeated washing and leads over time to fading of the coloration of the hair. The use of commercial rinse-out and leave-in care products does not sufficiently improve the fastness of the artificial color of hair.

It is thus desirable to develop methods for protecting the artificial color of keratin fibers from the effects of repeated washing.

The present inventors have discovered, surprisingly and unexpectedly, that the use of least one unsaturated fatty substance of formula (I) as defined herein can provide protection of the artificial color of keratin fibers against washing.

As used herein, the term “human keratin fibers” means head hair, body hair such as the beard and moustache, the eyelashes, and the eyebrows.

As used herein, the term “artificially dyed keratin fibers” means keratin fibers dyed via a direct dyeing process or via an oxidation dyeing process.

As used herein, the term “washing” means at least one application onto the keratin fibers of an aqueous rinse-out composition, which can be a detergent composition such as a shampoo. This term also includes bathing, for example, in the sea or in a swimming pool.

Disclosed herein is also a process for protecting color with respect to the washing of artificially dyed keratin fibers, comprising applying to the fibers at least one composition comprising, in a cosmetically acceptable medium, at least one unsaturated fatty substance of formula (I) as defined herein.

In at least one embodiment, the protection provided by the treatment according to the present disclosure may be long-lasting, i.e. does not require frequent reapplication of the product.

Also disclosed herein is a dyeing process comprising applying to keratin fibers, for example, human keratin fibers such as the hair, a direct or oxidation dye composition (A) for a time sufficient to develop the color, and applying a composition (B) comprising, in a cosmetically acceptable medium, at least one unsaturated fatty substance of formula (I) as defined herein, wherein composition (B) may be applied before or after the application of composition (A).

Other characteristics, aspects, subjects and benefits of the invention will be understood more clearly upon reading the description below. All meanings and definitions of the compounds used in the present disclosure given herein are valid for all embodiments of the present disclosure.

The unsaturated fatty substances in accordance with the present disclosure are chosen from those of formula (I): 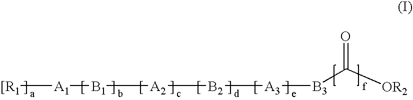
wherein:

A₁, A₂, and A₃, which may be identical or different, are chosen from linear and cyclic, monovalent and divalent hydrocarbon-based radicals comprising at least one unsaturation;

B₁, B₂, and B₃, which may be identical or different, are chosen from CₙH₂ₙ radicals in which n is an integer less than 20;

R₁ is chosen from linear and branched C₁-C₁₂ alkyl radicals;

R₂ is chosen from hydrogen, alkali metals M, and sorbitan groups of formula:

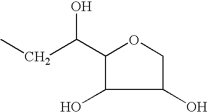

a, b, c, d, e, and f, which may be identical or different, are equal to 0 or 1.

As used herein, the term “unsaturation” means a double or triple bond.

In at least one embodiment, the divalent radical unsaturations are in a conformation chosen from cis and trans conformations. In another embodiment, the divalent radical is in cis conformation.

In yet another embodiment, M is chosen from Na⁺ and K⁺.

According to a further embodiment, the compounds of formula (I) can be chosen from compounds of formula (II): 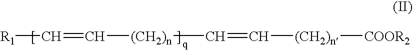
wherein:

R₁ is chosen from linear and branched C₁-C₁₂ alkyl radicals;

R₂ is chosen from hydrogen, alkali metals, and sorbitan groups of formula:

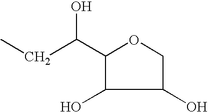

n and n′, which may be identical or different, are integers ranging from 1 to 10 and, in at least one embodiment, from 1 to 8; and

q is an integer ranging from 0 to 2.

According to another embodiment, the compounds of formula (II) may be chosen, for example, from:
lauroleic acid;
myristoleic acid;
palmitoleic acid;
oleic acid;
linoleic acid;
linolenic acid; and
sorbitan oleate;
   and in at least one embodiment, chosen from oleic acid and sorbitan oleate.

The cosmetically acceptable medium of the color-protecting compositions according to the present disclosure may be chosen, for example, from water and mixtures of water and at least one cosmetically acceptable organic solvent. Examples of suitable organic solvents include, but are not limited to, C₁-C₄ lower alkanols, such as ethanol and isopropanol; polyols and polyol ethers, for instance 2-butoxyethanol, propylene glycol, propylene glycol monomethyl ether, and diethylene glycol monoethyl ether and monomethyl ether, and mixtures thereof.

The at least one solvent may be present in the composition in an amount ranging from 1% to 40% by weight, for example, from 3% to 10% by weight, relative to the total weight of the composition.

The at least one unsaturated fatty substance of formula (I) is present in the color-protecting compositions in an amount ranging from 1% to 100%, for example, from 5% to 90% by weight relative to the total weight of the composition.

According to one embodiment of the present disclosure, a composition comprising 100% by weight of at least one unsaturated fatty substance of formula (I) may be used. In this embodiment, the physiologically acceptable medium will consist of the at least one unsaturated fatty substance of formula (I).

The composition according to the present disclosure comprising the at least one agent for protecting the color of keratin fibers may also comprise at least one adjuvant chosen from various adjuvants conventionally used in hair treatment compositions, such as anionic, cationic, nonionic, amphoteric, and zwitterionic surfactants, and mixtures thereof; anionic, cationic, nonionic, amphoteric, and zwitterionic polymers, and mixtures thereof; mineral and organic thickeners, such as anionic, cationic, nonionic, and amphoteric polymeric associative thickeners; penetrants; sequestrants; fragrances; buffers; dispersants; conditioning agents, for instance, modified and unmodified, volatile and non-volatile silicones; film-forming agents; ceramides; preserving agents; and opacifiers.

According to one embodiment, the compositions according to the present disclosure may also comprise at least one agent for protecting against the effects of atmospheric agents, such as light.

The at least one agent for protecting keratin fibers against atmospheric agents may be any active agent that is useful for preventing or limiting the degradation of keratin fibers, such as the hair, caused by atmospheric attacking factors, for instance, light.

Thus, this agent for protecting keratin fibers may be chosen, for example, from organic UV-screening agents, free-radical scavengers, and antioxidants.

As used herein, the term “free-radical scavenger” means any compound capable of trapping free radicals.

The organic UV-screening agents (systems for screening out UV radiation) may be chosen, for instance, from water-soluble or liposoluble, silicone or non-silicone screening agents.

By way of non-limiting example, the organic UV-screening agents may be chosen from dibenzoylmethane derivatives; anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzazolyl derivatives as described, for instance, in European Patent No. 0 669 323 and U.S. Pat. No. 2,463,264; p-aminobenzoic acid (PABA) derivatives; benzoxazole derivatives as described, for example, in European Patent Application Nos. 0 832 642, 1 027 883, and 1 300 137 and German Patent Application No. 101 62 844; screening polymers and screening silicones such as those described in International Patent Application Publication No. WO 93/04665; dimers derived from α-alkylstyrene, such as those described in German Patent Application No. 198 55 649; 4,4-diarylbutadienes such as those described in European Patent Application Nos. 0 967 200, 1 008 586, 1 133 980, and 0 133 981, and German Patent Application Nos. 197 46 654 and 197 55 649, and mixtures thereof.

Other examples of organic UV-screening agents include, but are not limited to, those denoted hereinbelow under their INCI name, and mixtures thereof:

Para-Aminobenzoic Acid Derivatives:

PABA,

Ethyl PABA,

Ethyl dihydroxypropyl PABA,

Ethylhexyl dimethyl PABA sold, for example, under the name “Escalol 507” by ISP, Glyceryl PABA, and

PEG-25 PABA sold under the name “Uvinul P25” by BASF.

Cinnamic Derivatives:

Ethylhexyl methoxycinnamate sold, for instance, under the trade name “Parsol MCX” by Hoffmann LaRoche,

Isopropyl methoxycinnamate,

Isoamyl methoxycinnamate sold, for example, under the trade name “Neo Heliopan E 1000” by Haarmann and Reimer,

Cinoxate,

DEA methoxycinnamate,

Diisopropyl methylcinnamate, and

Glyceryl ethylhexanoate dimethoxycinnamate.

Dibenzoylmethane Derivatives:

Butylmethoxydibenzoylmethane sold, for instance, under the trade name “Parsol 1789” by Hoffmann LaRoche, and

Isopropyldibenzoylmethane sold, for example, under the trade name “Eusolex 8020” by Merck.

Salicylic Derivatives:

Homosalate sold, for instance, under the name “Eusolex HMS” by Rona/EM Industries, Ethylhexyl salicylate sold, for example, under the name “Neo Heliopan OS” by Haarmann and Reimer,

Dipropylene glycol salicylate sold, for instance, under the name “Dipsal” by Scher, and TEA salicylate sold, for example, under the name “Neo Heliopan TS” by Haarmann and Reimer.

β,β-Diphenylacrylate Derivatives:

Octocrylene sold, for instance, under the trade name “Uvinul N539” by BASF, and Etocrylene sold, for example, under the trade name “Uvinul N35” by BASF.

Benzophenone Derivatives:

Benzophenone-1 sold, for instance, under the trade name “Uvinul 400” by BASF,

Benzophenone-2 sold, for example, under the trade name “Uvinul D50” by BASF,

Benzophenone-3 or Oxybenzone sold, for instance, under the trade name “Uvinul M40” by BASF,

Benzophenone-4 sold, for example, under the trade name “Uvinul MS40” by BASF,

Benzophenone-5,

Benzophenone-6 sold, for instance, under the trade name “Helisorb 11” by Norquay,

Benzophenone-8 sold, for example, under the trade name “Spectra-Sorb UV-24” by American Cyanamid,

Benzophenone-9 sold, for instance, under the trade name “Uvinul DS-49” by BASF,

Benzophenone-12, and

n-hexyl 2-(4-diethylamino-2-hydroxybenzoyl)benzoate.

Benzylidenecamphor Derivatives:

3-Benzylidenecamphor sold, for example, under the name “Mexoryl SD” by Chimex,

4-Methylbenzylidenecamphor sold, for instance, under the name “Eusolex 6300” by Merck,

Benzylidenecamphorsulfonic acid sold, for example, under the name “Mexoryl SL” by Chimex,

Camphor benzalkonium methosulfate sold, for instance, under the name “Mexoryl SO” by Chimex,

Terephthalylidenedicamphorsulfonic acid sold, for example, under the name “Mexoryl SX” by Chimex, and

Polyacrylamidomethylbenzylidenecamphor sold, for instance, under the name “Mexoryl SW” by Chimex.

Phenylbenzimidazole Derivatives:

Phenylbenzimidazolesulfonic acid sold, for example, under the trade name “Eusolex 232” by Merck, and

Disodium phenyl dibenzimidazole tetrasulfonate sold, for instance, under the trade name “Neo Heliopan AP” by Haarmann and Reimer.

Phenylbenzotriazole Derivatives:

Drometrizole trisiloxane sold, for example, under the name “Silatrizole” by Rhodia Chimie, and

Methylenebis(benzotriazolyl)tetramethylbutylphenol sold, for instance, in solid form under the trade name “MIXXIM BB/100” by Fairmount Chemical, or in micronized form as an aqueous dispersion under the trade name “Tinosorb M” by Ciba Specialty Chemicals.

Triazine Derivatives:

Bis-Ethylhexyloxyphenol Methoxyphenyl Triazine sold, for example, under the trade name “Tinosorb S” by Ciba Geigy,

Ethylhexyltriazone sold, for instance, under the trade name “Uvinul T150” by BASF,

Diethylhexylbutamidotriazone sold, for example, under the trade name “Uvasorb HEB” by Sigma 3V, and

2,4,6-tris(diisobutyl 4′-aminobenzalmalonate)-s-triazine.

Anthranilic Derivatives:

Menthyl anthranilate sold, for instance, under the trade name “Neo Heliopan MA” by Haarmann and Reimer.

Imidazoline Derivatives:

Ethylhexyldimethoxybenzylidenedioxoimidazoline propionate.

Benzalmalonate Derivatives:

Polyorganosiloxane containing benzalmalonate functional groups, for instance,

Polysilicone-15, sold under the trade name “Parsol SLX” by Hoffmann LaRoche

4,4-Diarylbutadiene Derivatives:

1,1-Dicarboxy(2,2′-dimethylpropyl)-4,4-diphenylbutadiene

Benzoxazole Derivatives:

2,4-bis[5-(1-dimethylpropyl)benzoxazol-2-yl(4-phenyl)imino]-6-(2-ethylhexyl)imino-1,3,5-triazine sold, for example, under the name Uvasorb K2A by Sigma 3V.

Liposoluble (or lipophilic) organic UV-screening agents suitable for use in accordance with the present disclosure include, but are not limited to:

ethylhexyl methoxycinnamate,

butylmethoxydibenzoylmethane,

Homosalate,

ethylhexyl salicylate,

Octocrylene,

Benzophenone-3,

n-hexyl 2-(4-diethylamino-2-hydroxybenzoyl)benzoate,

4-methylbenzylidenecamphor,

ethylhexyl triazone,

bis-ethylhexyloxyphenol methoxyphenyl triazine,

diethylhexyl butamido triazone,

drometrizole trisiloxane,

Polysilicone-15,

1,1-dicarboxy(2,2′-dimethylpropyl)-4,4-diphenylbutadiene, and

2,4-bis[5-1(dimethylpropyl)benzoxazol-2-yl(4-phenyl)imino]-6-(2-ethylhexyl)imino-1,3,5-triazine.

Examples of water-soluble (or hydrophilic) organic UV-screening agents include, but are not limited to:

PABA,

PEG-25 PABA

benzylidenecamphorsulfonic acid,

camphorbenzalkonium methosulfate,

terephthalylidenedicamphorsulfonic acid,

phenylbenzimidazolesulfonic acid,

disodium phenylbenzimidazoletetrasulfonate,

Benzophenone-4, and

Benzophenone-5.

Free-radical scavengers that may be used in the composition according to the present disclosure include, by way of non-limiting example, besides certain antipollution agents mentioned above, vitamin E and derivatives thereof such as tocopheryl acetate; bioflavonoids; coenzyme Q10, and ubiquinone; certain enzymes, for instarice, catalase, superoxide dismutase and wheatgerm extracts containing it, lactoperoxidase, glutathione peroxidase, and quinone reductases; glutathione; benzylidenecamphor; benzylcyclanones; substituted naphthalenones; pidolates; phytanetriol; gamma-oryzanol; guanosine; lignans; and melatonin.

In at least one embodiment, the antioxidants may be chosen from phenols such as BHA (tert-butyl-4-hydroxyanisole), BHT (2,6-di-tert-butyl-p-cresol), and TBHQ (tert-butylhydroquinone), polyphenols such as proanthocyanidol oligomers and flavonoids, hindered amines known under the generic term HALS (Hindered Amine Light Stabilizer) such as tetraminopiperidine, erythorbic acid, polyamines such as spermine, cysteine, glutathione, superoxide dismutase, and lactoferrin.

According to another embodiment, the at least one agent for protecting keratin fibers against atmospheric attacking factors such as light will be chosen from organic UV-screening agents.

The at least one agent for protecting keratin fibers against atmospheric attacking factors such as light may be present in the composition in an amount ranging from 0.2% to 20% by weight relative to the total weight of the composition.

According to one embodiment, the compositions according to the present disclosure comprise at least one aromatic alcohol and at least one aromatic dicarboxylic acid.

As used herein, the term “aromatic alcohol” means any compound that is liquid at room temperature and atmospheric pressure, comprising at least one ring chosen from benzene and naphthalene rings and at least one alcohol functional group (OH) directly linked to the ring or linked to at least one substituent of the ring. In at least one embodiment, the alcohol functional group can be on a substituent of the at least one ring chosen from benzene and naphthalene rings.

Examples of aromatic alcohols that may be used in the composition according to the present disclosure include, but are not limited to:

benzyl alcohol,

benzoylisopropanol,

benzyl glycol,

phenoxyethanol,

dichlorobenzyl alcohol,

methylphenylbutanol,

phenoxyisopropanol,

phenylisohexanol,

phenylpropanol,

phenylethyl alcohol, and

mixtures thereof.

In at least one embodiment, the at least one aromatic alcohol is benzyl alcohol.

The at least one aromatic alcohol may be present in the composition in an amount ranging from 0.01% to 50% by weight, for example, from 0.1% to 30% by weight, or from 1% to 20% by weight, relative to the total weight of the composition. According to one embodiment, the at least one aromatic alcohol is present in the composition in an amount greater than 1% by weight.

The compositions according to the present disclosure may further comprise at least one optionally salified aromatic carboxylic acid.

As used herein, the term “aromatic carboxylic acid” means any compound comprising at least one ring chosen from benzene and naphthalene rings and at least one carboxylic acid functional group (COOH), in free or salified form, directly linked to the ring or linked to at least one substituent of the ring. In at least one embodiment, the acid functional group may be directly linked to the at least one ring chosen from benzene and naphthalene rings.

The aromatic carboxylic acid salts may be chosen, for example, from alkali metal (e.g, sodium and potassium) salts, alkaline-earth metal (e.g., calcium and magnesium) salts, organic amine salts, and ammonium salts.

Examples of aromatic carboxylic acids that may be used in the composition according to the present disclosure include, but are not limited to:

benzoic acid,

para-anisic acid,

diphenolic acid,

ferulic acid,

hippuric acid,

3-hydroxybenzoic acid,

4-hydroxybenzoic acid,

phenylthioglycolic acid,

acetylsalicylic acid,

para-, meta-, or ortho-phthalic acid,

the salified forms thereof, and

mixtures thereof.

According to one embodiment, the at least one aromatic carboxylic acid is benzoic acid.

The at least one optionally salified carboxylic aromatic acid may be present in the composition in an amount ranging from 0.001% to 30% by weight, for example, from 0.01% to 20% by weight, or from 0.1% to 10% by weight, relative to the total weight of the composition.

The compositions according to the present disclosure may further comprise at least one conditioning agent.

As used herein, the term “conditioning agent” means any agent whose function is to improve the cosmetic properties of the hair, for instance, the softness, disentangling, feel, smoothness, and/or static electricity.

The conditioning agents may be in liquid, semi-solid, or solid form, for example, oils, waxes, and gums.

The conditioning agents may be chosen, for example, from synthetic oils such as polyolefins, animal and plant oils, fluoro oils, perfluoro oils, natural waxes, synthetic waxes, silicones, non-polysaccharide cationic polymers, ceramide compounds, cationic surfactants, fatty amines, saturated fatty acids, esters of fatty acids other than those of the present disclosure, and mixtures thereof.

The synthetic oils may be chosen, in at least one embodiment, from polyolefins, for instance, poly-α-olefins such as:

those of hydrogenated or non-hydrogenated polybutene type, for instance, those of hydrogenated or non-hydrogenated polyisobutene type.

Isobutylene oligomers with a molecular weight of less than 1,000 and mixtures thereof with polyisobutylenes with a molecular weight of greater than 1,000, for example, ranging from 1,000 to 15,000, may also be used.

Non-limiting examples of poly-α-olefins that can be used in accordance with the present disclosure include the polyisobutenes sold under the name Permethyl 99 A, 101 A, 102 A, 104 A (n=16), and 106 A (n=38) by the company Presperse Inc., and the products sold under the name Arlamol HD (n=3) by the company ICI (n denoting the degree of polymerization), and

those of hydrogenated or non-hydrogenated polydecene type.

Such products are sold, for example, under the names Ethylflo by the company Ethyl Corp. and Arlamol PAO by the company ICI.

The animal and plant oils may be chosen, for instance, from sunflower oil, corn oil, soybean oil, avocado oil, jojoba oil, marrow oil, grapeseed oil, sesame oil, hazelnut oil, fish oils, glyceryl tricaprocaprylate, and plant and animal oils of formula R₉COOR₁₀, wherein R₉ is chosen from higher fatty acid residues comprising from 7 to 29 carbon atoms and R₁₀ is chosen from linear and branched hydrocarbon-based chains comprising from 3 to 30 carbon atoms, such as alkyl and alkenyl chains, for example, purcellin oil.

In at least one embodiment, natural and synthetic essential oils such as eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil, and bergamot oil may be used.

The waxes may be chosen from natural (animal and plant) and synthetic substances that are solid at room temperature (20°-25° C.). The waxes are also insoluble in water, soluble in oils, and are capable of forming a water-repellent film.

A suitable definition of waxes may be found, for example, in P. D. Dorgan, Drug and Cosmetic Industry, December 1983, pp. 30-33.

The at least one wax may be chosen, for example, from carnauba wax, candelilla wax, alfalfa wax, paraffin wax, ozokerite, plant waxes such as olive tree wax, rice wax, hydrogenated jojoba wax, absolute waxes of flowers such as the essential wax of blackcurrant flower sold by the company Bertin (France), animal waxes such as beeswaxes, and modified beeswaxes (cerabellina). Other examples of waxes and waxy starting materials which can be used according to the present disclosure include, but are not limited to, marine waxes such as the product sold by the company Sophim under the reference M82, and polyethylene waxes and polyolefin waxes in general.

According to one embodiment, the at least one conditioning agent may be chosen from cationic polymers and silicones.

Non-saccharide cationic polymers that may be used in accordance with the present disclosure may be chosen from those known in the art as improving the cosmetic properties of hair treated with detergent compositions, for example, those described in European Patent Application No. 0 337 354 and French Patent Application Nos. 2 270 846, 2 383 660, 2 598 611, 2 470 596, and 2 519 863.

As used herein, the term “non-saccharide polymers” is understood to mean polymers that do not contain a glycoside bond between monosaccharides.

As used herein, the term “cationic polymer” denotes any polymer containing cationic groups and/or groups that may be ionized into cationic groups.

The cationic polymers may be chosen from those comprising units comprising at least one group chosen from primary, secondary, tertiary, and/or quaternary amine groups that either may form part of the main polymer chain or may be borne by a side substituent directly attached thereto.

The cationic polymers may, in at least one embodiment, have a number-average molecular mass ranging from 500 to 5×10⁶, for example, from 10³ to 3×10⁶.

Non-limiting examples of suitable cationic polymers include polyamine polymers, polyamino amide polymers, and polyquaternary ammonium polymers, which are known in the art.

The polyamine polymers, polyamino amide polymers, and polyquaternary ammonium polymers that may be used in accordance with the present disclosure include, by way of non-limiting example, those described in French Patents Nos 2 505 348 and 2 542 997, such as:

(1) homopolymers and copolymers derived from acrylic and methacrylic esters and amides and comprising at least one unit chosen from those of formulae (III)-(VI): 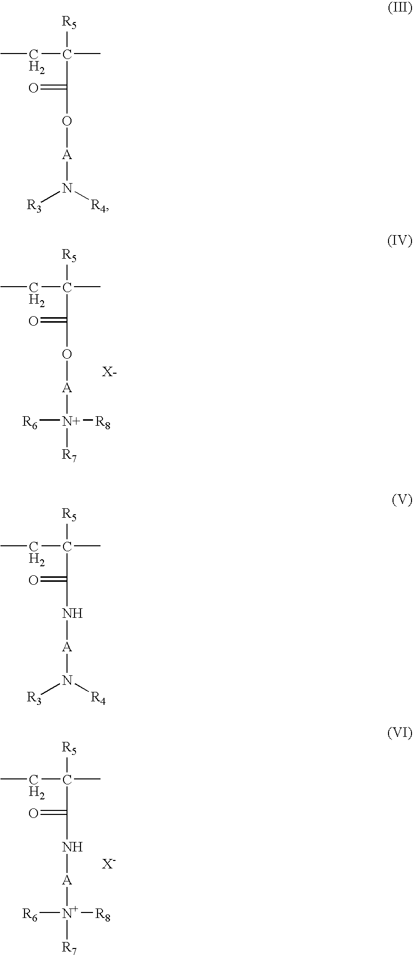
wherein:

R₃ and R₄, which may be identical or different, are chosen from hydrogen and alkyl groups comprising from 1 to 6 carbon atoms, for example, methyl and ethyl;

R₅, which may be identical or different, is chosen from hydrogen and CH₃ radicals;

A, which may be identical or different, is chosen from linear and branched alkyl groups comprising from 1 to 6 carbon atoms, for example, from 2 to 3 carbon atoms, and hydroxyalkyl groups comprising from 1 to 4 carbon atoms;

R₆, R₇, and R₈, which may be identical or different, are chosen from alkyl groups comprising from 1 to 18 carbon atoms and benzyl radicals and, in at least one embodiment, alkyl groups comprising from 1 to 6 carbon atoms; and

X is chosen from anions derived from acids chosen from mineral and organic acids, such as methosulfate anions and halides such as chloride and bromide.

The copolymers of family (1) may also comprise at least one unit derived from comonomers which may be chosen from acrylamides, methacrylamides, diacetone acrylamides, acrylamides and methacrylamides substituted on the nitrogen with lower (C₁-C₄) alkyls, acrylic and methacrylic acids and esters thereof, vinyllactams such as vinylpyrrolidone and vinylcaprolactam, and vinyl esters.

Examples of these copolymers of family (1) include, but are not limited to:

copolymers of acrylamide and of dimethylaminoethyl methacrylate quaternized with dimethyl sulfate or with a dimethyl halide, such as the product sold under the name Hercofloc by the company Hercules,

the copolymers of acrylamide and of methacryloyloxyethyltrimethylammonium chloride described, for example, in European Patent Application No. 0 080 976 and sold under the name Bina Quat P 100 by the company Ciba Geigy,

the copolymer of acrylamide and of methacryloyloxyethyltrimethylammonium methosulfate sold under the name Reten by the company Hercules,

quaternized or non-quaternized vinylpyrrolidone/dialkylaminoalkyl acrylate or methacrylate copolymers, such as the products sold under the name Gafquat by the company ISP, such as, for example, Gafquat 734 and Gafquat 755, and the products known as Copolymer 845, 958, and 937. Such polymers are described, for instance, in French Patent Nos. 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold, for example, under the name Styleze CC 10 by ISP, and

quaternized vinylpyrrolidone/dimethylaminopropylmethacrylamide copolymers, such as the product sold under the name Gafquat HS 100 by the company ISP.

(2) Polymers comprising at least one entity chosen from piperazinyl units and divalent alkylene and hydroxyalkylene radicals comprising straight or branched chains, optionally interrupted by at least one entity chosen from oxygen, sulphur, and nitrogen atoms, aromatic rings, and heterocyclic rings, as well as the oxidation and/or quaternization products of these polymers. Such polymers are described, for example, in French Patent Nos. 2 162 025 and 2 280 361.

(3) Water-soluble polyamino amides prepared by polycondensation of an acidic compound with a polyamine; these polyamino amides may optionally be crosslinked with at least one agent chosen from epihalohydrins, diepoxides, dianhydrides, unsaturated dianhydrides, bis-unsaturated derivatives, bis-halohydrins, bis-azetidiniums, bis-haloacyldiamines, bis-alkyl halides, and oligomers resulting from the reaction of a difunctional compound which is reactive with an agent chosen from bis-halohydrins, bis-azetidiniums, bis-haloacyldiamines, bis-alkyl halides, epihalohydrins, diepoxides, and bis-unsaturated derivatives; the crosslinking agent being used in an amount ranging from 0.025 to 0.35 mol per amine group of the polyamino amide; these polyamino amides possibly being alkylated or, if they contain at least one tertiary amine functional group, they may be quaternized. Such polymers are described, for example, in French Patent Nos. 2 252 840 and 2 368 508.

(4) The polyamino amide derivatives resulting from the condensation of polyalkylene polyamines with polycarboxylic acids followed by alkylation with difunctional agents, for example, adipic acid/dialkylaminohydroxyalkyldialkylenetriamine polymers wherein the alkyl radical comprises from 1 to 4 carbon atoms and, in at least one embodiment, is chosen from methyl, ethyl, and propyl. Such polymers are described, for example, in French Patent No. 1 583 363.

Non-limiting examples of these derivatives include, but are not limited to, the adipic acid/dimethylaminohydroxypropyl/diethylenetriamine polymers sold under the names Cartaretine F, F4, and F8 by the company Sandoz.

(5) The polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid may range from 0.8:1 to 1.4:1; the polyamino amide resulting therefrom being reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Polymers of this type are sold, for example, under the name Hercosett 57 by the company Hercules Inc. and under the name PD 170 or Delsette 101 by the company Hercules in the case of the adipic acid/epoxypropyl/diethylenetriamine copolymer.

(6) Cyclopolymers of alkyldiallylamine or of dialkyldiallylammonium, such as the homopolymers or copolymers containing, as main constituent of the chain, units chosen from those of formulae (VII) and (VIII): 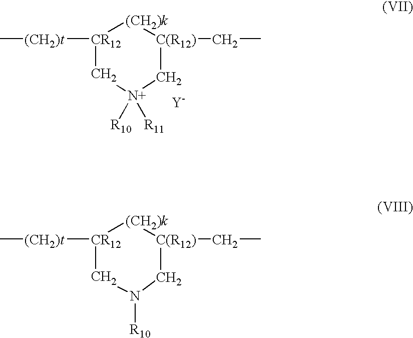
wherein:

k and t are equal to 0 or 1, the sum k+t being equal to 1;

R₁₂ is chosen from hydrogen and methyl radicals;

R₁₀ and R₁₁, which may be identical or different, are chosen from alkyl groups comprising from 1 to 6 carbon atoms, hydroxyalkyl groups in which the alkyl group comprises from 1 to 5 carbon atoms, and lower (C₁-C₄) amidoalkyl groups, or R₁₀ and R₁₁ may form, together with the nitrogen atom to which they are attached, heterocyclic groups such as piperidyl and morpholinyl; and

Y⁻ is an anion such as bromide, chloride, acetate, borate, citrate, tartrate, bisulfate, bisulfite, sulfate, and phosphate. These polymers are described, for example, in French Patent No. 2 080 759 and its Certificate of Addition 2 190 406.

In at least one embodiment, R₁₀ and R₁₁, which may be identical or different, are chosen from alkyl groups comprising from 1 to 4 carbon atoms.

Examples of such polymers include, but are not limited to, the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) The quaternary diammonium polymers comprising repeating units of formula (IX):

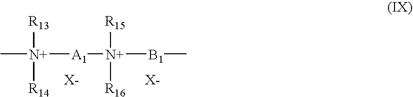

wherein:

R₁₃, R₁₄, R₁₅, and R₁₆, which may be identical or different, are chosen from aliphatic, alicyclic, and arylaliphatic radicals comprising from 1 to 20 carbon atoms and lower hydroxyalkylaliphatic radicals, or alternatively R₁₃, R₁₄, R₁₅, and R₁₆, may form, together or separately, with the nitrogen atoms to which they are attached, heterocycles optionally containing a second hetero atom other than nitrogen, or alternatively R₁₃, R₁₄, R₁₅, and R₁₆ may be chosen from linear and branched C₁-C₆ alkyl radicals substituted with at least one group chosen from nitrile groups, ester groups, acyl groups, amide groups, —CO—O—R₁₇-D groups, and —CO—NH—R₁₇-D groups, where R₁₇ is an alkylene radical and D is a quaternary ammonium group;

A₁ and B₁, which may be identical or different, are chosen from polymethylene groups comprising from 2 to 20 carbon atoms which may be linear or branched, saturated or unsaturated, and which may comprise, linked to or intercalated in the main chain, at least one entity chosen from aromatic rings, oxygen, sulphur, sulfoxide groups, sulfone groups, disulfide groups, amino groups, alkylamino groups, hydroxyl groups, quaternary ammonium groups, ureido groups, amide groups, and ester groups, and

X⁻ is an anion derived from a mineral or organic acid;

A₁, R₁₃, and R₁₅ may form, together with the two nitrogen atoms to which they are attached, a piperazine ring; in addition, if A₁ is chosen from linear or branched, saturated or unsaturated alkylene or hydroxyalkylene radicals, B₁ can also be chosen from (CH₂)ₙ—CO-D-OC—(CH₂)ₙ— groups, wherein:
n is an integer ranging from 2 to 20, and
D is chosen from:

a) glycol residues of formula: —O-Z-O—, wherein Z is chosen from linear and branched hydrocarbon-based radicals and groups of the following formulae:
—(CH₂—CH₂—O)ₓ—CH₂—CH₂—
—[CH₂—CH(CH₃)—O]_{y}—CH₂—CH(CH₃)—

wherein x and y, which may be identical or different, are chosen from integers ranging from 1 to 4, representing a defined and unique degree of polymerization, and numbers ranging from 1 to 4, representing an average degree of polymerization;

b) bis-secondary diamine residues such as a piperazine derivative;

c) bis-primary diamine residues of formula: —NH—Y—NH—, wherein Y is chosen from linear and branched hydrocarbon-based radicals and the divalent radical: —CH₂—CH₂—S—S—CH₂—CH₂—; and

d) ureylene groups of formula: —NH—CO—NH—.

In at least one embodiment, X⁻ is an anion chosen from chloride and bromide.

In another embodiment, these polymers can have a number-average molecular mass ranging from 1,000 to 100,000.

Polymers of this type are described, for example, in French Patent Nos. 2 320 330, 2 270 846, 2 316 271, 2 336 434, and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945, and 4,027,020.

In at least one embodiment, the cationic polymers may be chosen from those comprising at least one repeating unit of formula (a): 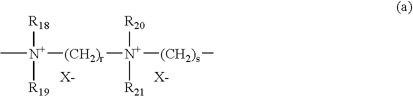
wherein R₁₈, R₁₉, R₂₀, and R₂₁, which may be identical or different, are chosen from alkyl and hydroxyalkyl radicals comprising from 1 to 4 carbon atoms, r and s are integers ranging from 2 to 20, and X⁻ is an anion derived from a mineral or organic acid.

In another embodiment, in formula (a), R₁₈, R₁₉, R₂₀, and R₂₁ are methyl radicals, r=3, s=6, and X═Cl; this compound is referred to as hexadimethrine chloride according to the INCI nomenclature (CTFA).

(8) Polyquaternary ammonium polymers comprising at least one unit of formula (X):

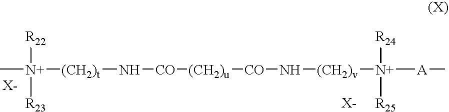

wherein:

R₂₂, R₂₃, R₂₄, and R₂₅, which may be identical or different, are chosen from hydrogen, methyl radicals, ethyl radicals, propyl radicals, β-hydroxyethyl radicals, β-hydroxypropyl radicals, and —CH₂CH₂(OCH₂CH₂)ₚOH radicals,

p is an integer ranging from 0 to 6, with the proviso that R₂₂, R₂₃, R₂₄, and R₂₅ are not simultaneously a hydrogen atom,

t and u, which may be identical or different, are integers ranging from 1 to 6,

v is an integer ranging from 0 to 34,

X⁻ is an anion such as a halide, and

A is a divalent radical and, in at least one embodiment, —CH₂—CH₂—O—CH₂—CH₂—.

Such compounds are described, for instance, in European Patent Application No. 0 122 324.

Examples of suitable commercial products corresponding to these polymers include, but are not limited to, Mirapol® A 15, Mirapol® AD1, Mirapol® AZ1 and Mirapol® 175 sold by the company Miranol.

(9) Quaternary polymers of vinylpyrrolidone and of vinylimidazole, for example, the products sold under the names Luviquat® FC 905, FC 550, and FC 370 by the company BASF.

(10) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example, methylenebisacrylamide. According to one embodiment, a crosslinked acrylamide/methacryloyloxyethyltrimethylammonium chloride copolymer (20/80 by weight) in the form of a dispersion containing 50% by weight of the copolymer in mineral oil may be used. This dispersion is sold under the name Salcare® SC 92 by the company Ciba. According to another embodiment, a crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Ciba.

Other examples of cationic polymers that can be used in accordance with the present disclosure include, but are not limited to, cationic proteins, cationic protein hydrolysates, polyalkyleneimines, such as polyethyleneimines, polymers comprising at least one unit chosen from vinylpyridine units and vinylpyridinium units, condensates of polyamines and of epichlorohydrin, quaternary polyureylenes, and chitin derivatives.

According to at least one embodiment, the cationic polymers may be chosen from cationic cyclopolymers, for example, the dimethyldiallylammonium chloride homopolymers and copolymers sold under the names Merquat 100, Merquat 550, and Merquat S by the company Nalco, quaternary vinylpyrrolidone and vinylimidazole polymers, and mixtures thereof.

Examples of silicones that may be used in accordance with the present disclosure include, but are not limited to, polyorganosiloxanes that are insoluble in the composition and that may be in the form of oils, waxes, resins, or gums.

Organopolysiloxanes are defined, for example, in Walter Noll's “Chemistry and Technology of Silicones” (1968) Academic Press. They can be volatile or non-volatile.

When they are volatile, the silicones may be chosen, for instance, from those having a boiling point ranging from 60° C. to 260° C., such as:

(i) cyclic silicones comprising from 3 to 7, for example, from 4 to 5 silicon atoms. These compounds include, for example, octamethylcyclotetrasiloxane, such as the products sold under the names Volatile Silicone 7207 by Union Carbide and Silbione 70045 V 2 by Rhodia Chimie, decamethylcyclopentasiloxane, such as the products sold under the names Volatile Silicone 7158 by Union Carbide and Silbione 70045 V 5 by Rhodia Chimie, and mixtures thereof.

Other non-limiting examples of cyclic silicones include cyclocopolymers of the dimethylsiloxanes/methylalkylsiloxane type, such as Volatile Silicone FZ 3109 sold by the company Union Carbide, having the chemical structure:

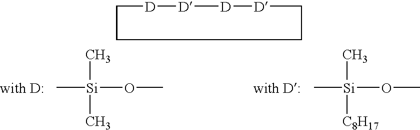

Mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane may also be used;

(ii) linear volatile silicones comprising from 2 to 9 silicon atoms and having a viscosity of less than or equal to 5×10⁻⁶ m²/s at 25° C. A non-limiting example of such silicones is decamethyltetrasiloxane sold, for instance, under the name SH 200 by the company Toray Silicone. Silicones belonging to this category are also described, for example, in the article published in Cosmetics and Toiletries, Vol. 91, Jan. 76, pp. 27-32, Todd & Byers “Volatile Silicone Fluids for Cosmetics.”

According to at least one embodiment, the silicones are chosen from non-volatile silicones, such as polyalkylsiloxanes, polyarylsiloxanes, polyalkylarylsiloxanes, silicone gums and resins, polyorganosiloxanes modified with organofunctional groups, and mixtures thereof.

In another embodiment, the silicones may be chosen from polyalkylsiloxanes, such as polydimethylsiloxanes comprising trimethylsilyl end groups having a viscosity ranging from 5×10⁻⁶ to 2.5 m²/s at 25° C., for example, from 1×10⁻⁵ to 1 m²/s. The viscosity of the silicones may be measured, for example, at 25° C. according to ASTM standard 445 Appendix C.

Non-limiting examples of suitable polyalkylsiloxanes include, but are not limited to:

the Silbione oils of the 47 and 70 047 series or the Mirasil oils sold by Rhodia Chimie, for example, the oil 70 047 V 500 000;

the oils of the Mirasil series sold by the company Rhodia Chimie;

the oils of the 200 series from the company Dow Corning, such as DC200 with a viscosity of 60,000 cSt; and

the Viscasil oils from General Electric and certain oils of the SF series (e.g., SF 96, and SF 18) from General Electric.

The polydimethylsiloxanes may also be chosen, for example, from those comprising dimethylsilanol end groups (Dimethiconol according to the CTFA name) such as the oils of the 48 series from the company Rhodia Chimie.

Other examples of polyalkylsiloxanes falling within this category include, but are hot limited to, the products sold under the names Abil Wax 9800 and 9801 by the company Goldschmidt, which are poly(C₁-C₂₀)alkylsiloxanes.

According to at least one embodiment, the polyalkylarylsiloxanes may be chosen from linear and/or branched polydimethylmethylphenylsiloxanes and polydimethyldiphenylsiloxanes having a viscosity ranging from 1×10⁻⁵ to 5×10⁻² m²/s at 25° C.

Polyalkylarylsiloxanes suitable for use in accordance with the present disclosure include, but are not limited to:

the Silbione oils of the 70 641 series from Rhodia Chimie;

the oils of the Rhodorsil 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250, and SF 1265.

The silicone gums that may be used in accordance with the present disclosure include, for example, polydiorganosiloxanes with high number-average molecular masses ranging from 200,000 to 1,000,000, used alone or as a mixture in a solvent. This solvent can be chosen, for instance, from volatile silicones, polydimethylsiloxane (PDMS) oils, polyphenylmethylsiloxane (PPMS) oils, isoparaffins, polyisobutylenes, methylene chloride, pentane, dodecane and tridecane, and mixtures thereof.

Further non-limiting examples of suitable silicone gums include:

polydimethylsiloxane,

polydimethylsiloxane/methylvinylsiloxane gums,

polydimethylsiloxane/diphonylsiloxane,

polydimethylsiloxane/phenylmethylsiloxane, and

polydimethylsiloxane/diphenylsiloxane/methylvinylsiloxane.

Mixtures of silicone gums may also be used, such as:

mixtures formed from a polydimethylsiloxane hydroxylated at the chain end (referred to as dimethiconols according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500,000, dissolved in the oil SF 1202 Silicone Fluid corresponding to decamethylcyclopentasiloxane;

mixtures of two PDMSs with different viscosities, for instance, of a PDMS gum and a PDMS oil, such as the product SF 1236 from the company General Electric. The product SF 1236 is a mixture of an SE 30 gum defined above, having a viscosity of 20 m² s, and an SF 96 oil, with a viscosity of 5×10⁻⁶ m²/s. In at least one embodiment, this product contains 15% SE 30 gum and 85% SF 96 oil.

The organopolysiloxane resins suitable for use in accordance with the present disclosure include, for instance, crosslinked siloxane systems comprising at least one unit chosen from:

R₂SiO_{2/2}, R₃SiO_{1/2}, RSiO_{3/2}, and SiO_{4/2}, wherein R is chosen from hydrocarbon-based groups comprising from 1 to 16 carbon atoms and phenyl groups. According to at least one embodiment, R may be chosen from C₁-C₄ lower alkyl radicals, such as methyl, and phenyl radicals.

Commercial products corresponding to these resins include, but are not limited to, the product sold under the name Dow Corning 593, those sold under the names Silicone Fluid SS 4230 and SS 4267 by the company General Electric, which are silicones of dimethyl/trimethyl siloxane structure, and the trimethyl siloxysilicate type resins sold, for example, under the names X22-4914, X21-5034, and X21-5037 by the company Shin-Etsu.

The organomodified silicones that can be used in accordance with the present disclosure include silicones as defined above and comprising in their structure at least one organofunctional group attached via a hydrocarbon-based radical.

Such organomodified silicones may include, for instance, polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 and the oils Silwet L 722, L 7500, L 77, and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, and the products sold under the names Q2 8220 and Dow Corning 929 and 939 by the company Dow Corning. According to one embodiment, the substituted amine groups may be chosen from C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and Abil Wax 2428, 2434 and 2440 by the company Goldschmidt;

hydroxylated groups such as the polyorganosiloxanes containing a hydroxyalkyl functional group, described, for example, in French Patent Application No. 85/16334, and chosen from those of formula (XI): 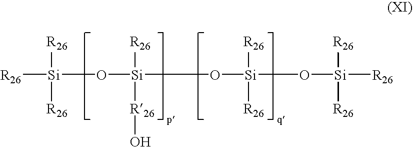
wherein the radicals R₂₆, which may be identical or different, are chosen from methyl and phenyl radicals; at least 60 mol % of the radicals R₂₆ denote methyl; the radical R′₂₆ is a C₂-C₁₈ divalent hydrocarbon-based alkylene chain unit; p′ is a number ranging from 1 to 30; and q′ is a number ranging from 1 to 150;

acyloxyalkyl groups, for example, the polyorganosiloxanes described in U.S. Pat. No. 4,957,732 and chosen from those of formula (XII): 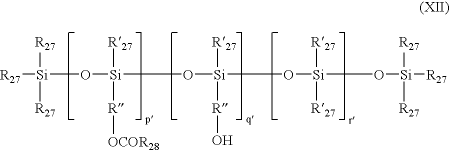
wherein:

R₂₇ is chosen from methyl, phenyl, —OCOR₂₈, and hydroxyl groups, wherein one of the radicals R₂₇ per silicon atom is possibly an OH group;

R′₂₇ is chosen from methyl and phenyl groups; wherein at least 60 mol % of all the radicals R₄ and R′₄ are methyl;

R₂₈ is chosen from C₈-C₂₀ alkyl and alkenyl radicals;

R″ is chosen from C₂-C₁₈ linear and branched divalent hydrocarbon-based alkylene radicals;

r′ is a number ranging from 1 to 120;

p′ is a number ranging from 1 to 30; and

q′ is equal to 0 or is less than 0.5 p′, the sum p′+q′ ranging from 1 to 30;
wherein the polyorganosiloxanes of formula (XII) may comprise groups: 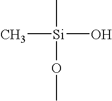
in an amount not exceeding 15% of the sum p+q+r;

anionic groups of carboxylic type, for example, in the products described in European Patent No. 0 186 507 from the company Chisso Corporation, and of alkylcarboxylic type, such as those present in the product X-22-3701E from the company Shin-Etsu; 2-hydroxyalkyl sulfonate; and 2-hydroxyalkyl thiosulfate such as the products sold by the company Goldschmidt under the names Abil S201 and Abil S255; and

hydroxyacylamino groups, such as the polyorganosiloxanes described in European Patent Application No. 342 834, and the product Q2-8413 from the company Dow Corning.

According to one embodiment, the silicones may be chosen from those comprising a polysiloxane portion and a portion comprising a non-silicone organic chain, one of the two portions constituting the main chain of the polymer, the other being grafted onto the main chain. These polymers are described, for example, in European Patent Application Nos. 0 412 704, 0 412 707, 0 640 105, and 0 582 152, International Patent Application Publication Nos. WO 95/00578 and WO 93/23009, and U.S. Pat. Nos. 4,693,935, 4,728,571, and 4,972,037. These polymers may be chosen from anionic and nonionic polymers.

Such polymers may be chosen, for example, from copolymers that can be obtained by free-radical polymerization starting with a monomer mixture comprising:

a) 50 to 90% by weight of tert-butyl acrylate;

b) 0 to 40% by weight of acrylic acid; and

c) 5 to 40% by weight of silicone macromer of formula (XIII): 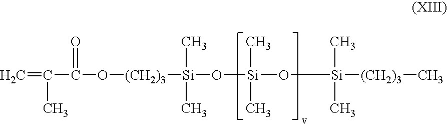
wherein v is a number ranging from 5 to 700; the weight percentages being calculated relative to the total weight of the monomers.

Other examples of grafted silicone polymers include, but are not limited to, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl(meth)acrylate type.

According to the present disclosure, the silicones disclosed herein may be used in a form chosen from emulsions, nanoemulsions, and microemulsions.

According to at least one embodiment, the polyorganosiloxanes may be chosen, for instance, from:

non-volatile silicones chosen from polyalkylsiloxanes comprising trimethylsilyl end groups, such as oils having a viscosity ranging from 0.2 to 2.5 m²/s at 25° C., such as the oils of the DC200 series from Dow Corning, for example, those with a viscosity of 60,000 cSt, of the Silbione 70047 and 47 series, such as the oil 70 047 V 500 000, which are sold by the company Rhodia Chimie, polyalkylsiloxanes comprising dimethylsilanol end groups, such as dimethiconols, and polyalkylarylsiloxanes such as the oil Silbione 70641 V 200 sold by the company Rhodia Chimie;

the organopolysiloxane resin sold under the name Dow Corning 593; and

polysiloxanes comprising amine groups, such as amodimethicones and trimethylsilylamodimethicones.

The cationic proteins and cationic protein hydrolysates may be chosen, for example, from chemically modified polypeptides bearing quaternary ammonium groups at the end of the chain or grafted thereto. Their molecular mass may range, for example, from 1,500 to 10,000, such as from 2,000 to 5,000. Examples of such compounds include, but are not limited to:

collagen hydrolysates bearing triethylammonium groups, such as the products sold under the name Quat-Pro E by the company Maybrook and referred to in the CTFA dictionary as “Triethonium Hydrolyzed Collagen Ethosulfate;”

collagen hydrolysates bearing trimethylammonium and trimethylstearylammonium chloride groups, sold under the name Quat-Pro S by the company Maybrook and referred to in the CTFA dictionary as “Steartrimonium Hydrolyzed Collagen;”

animal protein hydrolysates bearing trimethylbenzylammonium groups such as the products sold under the name Crotein BTA by the company Croda and referred to in the CTFA dictionary as “Benzyltrimonium hydrolyzed animal protein;” and

protein hydrolysates bearing, on the polypeptide chain, quaternary ammonium groups comprising at least one alkyl radical comprising from 1 to 18 carbon atoms.

Non-limiting examples of protein hydrolysates include various products sold by the company Croda, such as:

Croquat L in which the quaternary ammonium groups contain a C₁₂ alkyl group;

Croquat M in which the quaternary ammonium groups contain C₁₀-C₁₈ alkyl groups;

Croquat S in which the quaternary ammonium groups contain a C₁₈ alkyl group; and

Crotein Q in which the quaternary ammonium groups contain at least one alkyl group comprising from 1 to 18 carbon atoms.

Other examples of quaternized proteins and hydrolysates include, but are not limited to, those of formula (XIV): 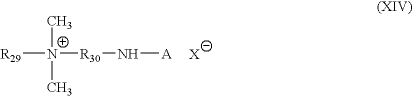
wherein:

X⁻ is an anion of an organic or mineral acid,

A is a protein residue derived from hydrolysates of collagen protein,

R₂₉ is chosen from lipophilic groups comprising up to 30 carbon atoms, and

R₃₀ is chosen from alkylene groups comprising from 1 to 6 carbon atoms. Commercial products corresponding to these compounds include, for example, the products sold by the company Inolex under the name Lexein QX 3000, referred to in the CTFA dictionary as “Cocotrimonium Collagen Hydrolysate.”

Further examples include, but are not limited to, quaternized plant proteins such as wheat, corn, and soybean proteins. Suitable quaternized wheat proteins include, for example, those sold by the company Croda under the names Hydrotriticum WQ and QM, referred to in the CTFA dictionary as “Cocodimonium Hydrolysed Wheat Protein,” Hydrotriticum QL, referred to in the CTFA dictionary as “Lauridimonium Hydrolysed Wheat Protein” and Hydrotriticum QS, referred to in the CTFA dictionary as “Steardimonium Hydrolysed Wheat Protein.”

According to the present disclosure, the compounds of ceramide type may be chosen, for instance, from natural and synthetic ceramides, glycoceramides, pseudoceramides, and/or neoceramides.

Suitable ceramide compounds are described, for example, in German Patent Application Nos. 4 424 530, 4 424 533, 4 402 929, and 4 420 736, International Patent Application Publication Nos. WO 95/23807, WO 94/07844, WO 95/16665, WO 94/07844, WO 94/24097, and WO 94/10131, European Patent Application Nos. 0 646 572 and 0 227 994, and French Patent Application No. 2 673 179, all of which are incorporated herein by reference in their entireties.

Further examples of ceramide compounds include, but are not limited to:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, such as N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine, and
mixtures thereof.

The at least one conditioning agent may also be chosen from cationic surfactants, such as optionally polyoxyalkylenated primary, secondary, and tertiary fatty amine salts; quaternary ammonium salts; imidazoline derivatives; and amine oxides of cationic nature.

Examples of suitable quaternary ammonium salts include, but are not limited to:

those of formula (XV): 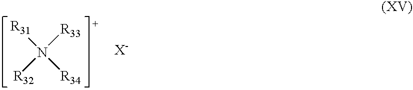
wherein the radicals R₃₁ to R₃₄, which may be identical or different, are chosen from linear and branched aliphatic radicals comprising from 1 to 30 carbon atoms and aromatic radicals such as aryl and alkylaryl. The aliphatic radicals may comprise hetero atoms such as oxygen, nitrogen, sulfur, and halogens. The aliphatic radicals may be chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate, and hydroxyalkyl radicals, comprising from 1 to 30 carbon atoms; and X⁻ is an anion chosen from halides, phosphates, acetates, lactates, (C₂-C₆)alkyl sulfates, alkyl sulfonates, and alkylaryl sulfonates;

quaternary ammonium salts of imidazolinium, for example, salts of formula (XVI): 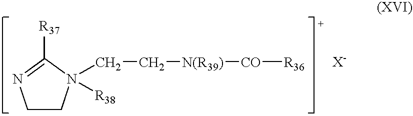
wherein:

R₃₆ is chosen from alkenyl and alkyl radicals comprising from 8 to 30 carbon atoms, for example, tallow fatty acid derivatives,

R₃₇ is chosen from hydrogen, C₁-C₄ alkyl radicals, and alkenyl and alkyl radicals comprising from 8 to 30 carbon atoms,

R₃₈ is chosen from C₁-C₄ alkyl radicals,

R₃₉ is chosen from hydrogen and C₁-C₄ alkyl radicals, and

X⁻ is an anion chosen from halides, phosphates, acetates, lactates, alkyl sulfates, alkyl sulfonates, and alkylaryl sulfonates. In at least one embodiment, R₃₆ and R₃₇ may be chosen from mixtures of alkenyl and alkyl radicals comprising from 12 to 21 carbon atoms, for example tallow fatty acid derivatives, R₃₈ is methyl, and R₃₉ is hydrogen. Such a product is sold, for example, under the name “Rewoquat W 75” by the company Degussa;

diquaternary ammonium salts of formula (XVII): 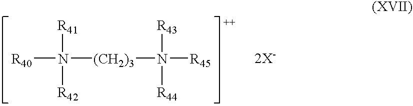
wherein:

R₄₀ is chosen from aliphatic radicals comprising from 16 to 30 carbon atoms,

R₄₁, R₄₂, R₄₃, R₄₄, and R₄₅, which may be identical or different, are chosen from hydrogen and alkyl radicals comprising from 1 to 4 carbon atoms, and

X⁻ is an anion chosen from halides, acetates, phosphates, nitrates, and methyl sulfates. A non-limiting example of such diquaternary ammonium salts is propane tallow diammonium dichloride;

quaternary ammonium salts comprising at least one ester functional group.

The quaternary ammonium salts comprising at least one ester functional group that may be used according to the present disclosure include, for example, those of formula (XVIII): 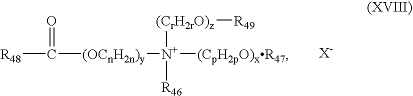
wherein:

R₄₆ is chosen from C₁-C₆ alkyl radicals and C₁-C₆ hydroxyalkyl and dihydroxyalkyl radicals;

R₄₇ is chosen from: 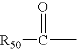
radicals,
linear and branched, saturated and unsaturated C₁-C₂₂ hydrocarbon-based radicals R₅₁, and
hydrogen,

R₄₉ is chosen from: 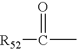
radicals,
linear and branched, saturated and unsaturated C₁-C₆ hydrocarbon-based radicals R₅₃, and
hydrogen,

R₄₈, R₅₀, and R₅₂, which may be identical or different, are chosen from linear and branched, saturated and unsaturated C₇-C₂₁ hydrocarbon-based radicals;

n, p, and r, which may be identical or different, are integers ranging from 2 to 6;

y is an integer ranging from 1 to 10;

x and z, which may be identical or different, are integers ranging from 0 to 10; and

X⁻ is chosen from simple and complex, organic and inorganic anions;

with the provisos that the sum x+y+z ranges from 1 to 15, that when x is 0, then R₄₇ denotes R₅₁, and that when z is 0, then R₄₉ denotes R₅₃.

According to one embodiment, the R₄₆ alkyl radicals may be chosen from linear and branched radicals and, in at least one other embodiment, linear radicals.

According to another embodiment, R₄₆ may be chosen from methyl, ethyl, hydroxyethyl, and dihydroxypropyl radicals and, in at least one embodiment, methyl and ethyl radicals.

According to yet another embodiment, the sum x+y+z may range from 1 to 10.

In a further embodiment, when R₄₇ is a hydrocarbon-based radical R₅₁, it may be long and comprise from 12 to 22 carbon atoms, or short and comprise from 1 to 3 carbon atoms.

According to a still further embodiment, when R₄₉ is a hydrocarbon-based radical R₅₃, it may comprise from 1 to 3 carbon atoms.

In another embodiment, R₄₈, R₅₀, and R₅₂, which may be identical or different, can be chosen from linear and branched, saturated and unsaturated C₁₁-C₂₁ hydrocarbon-based radicals and, in at least one embodiment, linear and branched, saturated and unsaturated, C₁₁-C₂₁ alkyl and alkenyl radicals.

According to yet another embodiment, x and z, which may be identical or different, can be equal to 0 or 1.

In a further embodiment, y may be equal to 1.

In a still further embodiment, n, p, and r, which may be identical or different, can be equal to 2 or 3 and, in at least one embodiment, equal to 2.

According to another embodiment, the anion X⁻ can be chosen from halides (e.g., chloride, bromide, and iodide), and alkyl sulfates, such as methyl sulfate. In yet another embodiment, the anion X⁻ can be chosen from methanesulfonate, phosphate, nitrate, tosylate, anions derived from an organic acid, such as acetate and lactate, and any other anion that is compatible with the ammonium comprising an ester functional group.

According to a further embodiment, the anion X⁻ can be chosen from chloride and methyl sulfate.

In at least one embodiment, the ammonium salts can be chosen from those of formula (XVIII) wherein:

R₄₆ is chosen from methyl and ethyl radicals,

x and y are equal to 1;

z is equal to 0 or 1;

n, p, and r are equal to 2;

R₄₇ is chosen from: 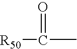
radicals,
methyl, ethyl, and C₁₄-C₂₂ hydrocarbon-based radicals; and
hydrogen;

R₄₉ is chosen from: 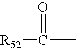
radicals, and
hydrogen;

R₄₈, R₅₀, and R₅₂, which may be identical or different, are chosen from linear and branched, saturated and unsaturated C₁₃-C₁₇ hydrocarbon-based radicals, for example, linear and branched, saturated and unsaturated C₁₃-C₁₇ alkyl and alkenyl radicals.

According to one embodiment, the hydrocarbon-based radicals are linear.

Further non-limiting examples include the compounds of formula (XVI) such as diacyloxyethyldimethylammonium salts, diacyloxyethylhydroxyethylmethylammonium salts, monoacyloxyethyldihydroxyethylmethylammonium salts, triacyloxyethylmethylammonium salts, monoacyloxyethylhydroxyethyldimethylammonium salts, and mixtures thereof. In at least one embodiment, these salts may be chosen from chloride and methyl sulfate salts. According to another embodiment, the acyl radicals can comprise from 14 to 18 carbon atoms and can be obtained from a plant oil such as palm oil and sunflower oil. When the compound contains several acyl radicals, these radicals may be identical or different.

These products can be obtained, for example, by direct esterification of an amine chosen from triethanolamine, triisopropanolamine, alkyldiethanolamines, and alkyldiisopropanolamines, which are optionally oxyalkylenated, with fatty acids or with fatty acid mixtures of plant or animal origin, or by transesterification of the methyl esters thereof. This esterification is followed by a quaternization using an alkylating agent such as alkyl halides (e.g., methyl and ethyl halides), dialkyl sulfates (e.g., dimethyl and diethyl sulfates), methyl methanesulfonate, methyl para-toluenesulfonate, glycol chlorohydrin, and glycerol chlorohydrin.

Such compounds are sold, for example, under the names Dehyquart by the company Cognis, Stepanquat by the company Stepan, Noxamium by the company CECA, and Rewoquat WE 18 by the company Degussa.

According to another embodiment, the ammonium salts comprising at least one ester functional group that are described in U.S. Pat. Nos. 4,874,554 and 4,137,180 may also be used.

In a further embodiment, the quaternary ammonium salts may be chosen from those of formula (XV), for example, tetraalkylammonium chlorides such as dialkyldimethylammonium chlorides and alkyltrimethylammonium chlorides, in which the alkyl radical comprises from 12 to 22 carbon atoms, for instance, behenyltrimethylammonium chloride, distearyldimethylammonium chloride, cetyltrimethylammonium chloride, and benzyldimethylstearylammonium chloride; and the stearamidopropyldimethyl(myristyl acetate)ammonium chloride sold under the name Ceraphyl 70 by the company Van Dyk.

In at least one embodiment, the fatty acids may be chosen, for example, from myristic acid, palmitic acid, stearic acid, behenic acid, and isostearic acid.

According to another embodiment, the fatty acid esters may be chosen from carboxylic acid esters, such as mono-, di-, tri-, and tetracarboxylic esters.

Examples of monocarboxylic acid esters include, but are not limited to, linear or branched, saturated or unsaturated C₁-C₂₆ aliphatic acid monoesters of linear or branched, saturated or unsaturated, C₁-C₂₆ aliphatic alcohols, the total carbon number of these esters being greater than or equal to 10.

Non-limiting examples of monoesters include dihydroabietyl behenate; octyidodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyidodecyl erucate; oleyl erucate; ethyl and isopropyl palmitates, 2-ethylhexyl palmitate, 2-octyidecyl palmitate, alkyl myristates such as isopropyl, butyl, cetyl, and 2-octyidodecyl myristate, hexyl stearate, butyl stearate, isobutyl stearate; dioctyl malate, hexyl laurate, 2-hexyldecyl laurate, isostearyl neopentanoate, and isodecyl neopentanoate.

According to one embodiment, C₄-C₂₂ di- and tricarboxylic acid esters of C₁-C₂₂ alcohols and mono-, di-, and tricarboxylic acid esters of C₂-C₂₆ di-, tri-, tetra-, and pentahydroxy alcohols may also be used.

Examples of such esters include, but are not limited to: diethyl sebacate; diisopropyl sebacate; diisopropyl adipate; di-n-propyl adipate; dioctyl adipate; diisostearyl adipate; dioctyl maleate; glyceryl undecylenate; octyldodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyldodecyl citrate; and trioleyl citrate.

In at least one embodiment, the esters may be chosen from ethyl and isopropyl palmitates, 2-ethylhexyl palmitate, 2-octyldecyl palmitate, alkyl myristates such as isopropyl, butyl, cetyl, and 2-octyldodecyl myristate, hexyl stearate, butyl stearate, isobutyl stearate; dioctyl malate, hexyl laurate, 2-hexyldecyl laurate, isononyl isononanoate, cetyl octanoate, isostearyl neopentanoate, and isodecyl neopentanoate.

The fluoro oils may be chosen, for example, from the perfluoropolyethers described in European Patent Application No. 0 486 135 and the fluorohydrocarbon compounds described in International Patent Application Publication No. WO 93/11103, which are incorporated herein by reference in their entireties.

As used herein, the term “fluorohydrocarbon compounds” denotes compounds whose chemical structure contains a carbon skeleton in which certain hydrogen atoms have been replaced with fluorine atoms.

The fluoro oils may also be chosen, for instance, from fluorocarbons such as fluoroamines, for example perfluorotributylamine, fluorohydrocarbons, for example perfluorodecahydronaphthalene, fluoro esters, and fluoro ethers.

Suitable perfluoropolyethers are sold, for example, under the trade names Fomblin by the company Montefluos and Krytox by the company Du Pont.

The fluorohydrocarbon compounds, may also be chosen from fluorine-containing fatty acid esters such as the product sold under the name Nofable FO by the company Nippon Oil.

It is to be understood that the compositions of the present disclosure may also include mixtures of any of the conditioning agents described above.

The at least one conditioning agent may be present in the composition in an amount ranging from 0.001% to 20% by weight, for example, from 0.01% to 10% by weight, or from 0.1% to 3% by weight, relative to the total weight of the final composition.

The compositions for protecting the color of keratin fibers according to the present disclosure may be in a form chosen from aqueous and aqueous-alcoholic haircare lotions, gels, milks, creams, emulsions, and mousses.

The compositions for protecting the color of keratin fibers may be packaged in various forms, for instance, in vaporizers, pump-dispenser bottles, and in aerosol containers in order to apply the composition in vaporized form or in the form of a mousse. Such packaging forms are indicated, for example, when it is desired to obtain a spray, a lacquer, or a mousse for treating the hair.

The pH of the composition for protecting the color of keratin fibers can range from 1 to 11, for example, from 2 to 6, and may be adjusted to the desired value via at least one acidifying or basifying agent that are known in the art for compositions applied to keratin fibers.

Examples of suitable basifying agents include, but are not limited to, aqueous ammonia, alkali metal carbonates, alkanolamines such as monoethanolamine, diethanolamine, triethanolamine, and derivatives thereof, oxyethylenated and/or oxypropylenated hydroxyalkylamines and ethylenediamines, sodium hydroxide, potassium hydroxide, and compounds of the formula: 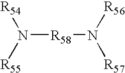
wherein R₅₈ is a propylene residue optionally substituted with at least one entity chosen from hydroxyl groups and C₁-C₄ alkyl radicals; and R₅₄, R₅₅, R₅₆, and R₅₇, which may be identical or different, are chosen from hydrogen, C₁-C₄ alkyl radicals, and C₁-C₄ hydroxyalkyl radicals.

Non-limiting examples of acidifying agents include, mineral and organic acids, for instance, hydrochloric acid, orthophosphoric acid, carboxylic acids, for instance, tartaric acid, citric acid, and lactic acid, and sulfonic acids.

Disclosed herein is a process for protecting the color of artificially dyed keratin fibers with respect to washing, comprising applying to the fibers, before or after dyeing, at least one composition comprising, in a cosmetically acceptable medium, at least one unsaturated fatty substance of formula (I) as defined herein.

In at least one embodiment, the composition comprising the at least one unsaturated fatty substance of formula (I) is applied to the fibers after the dyeing step.

The process may further comprise rinsing and/or washing with shampoo before and/or after the application of the composition containing the at least one unsaturated fatty substance of formula (I).

The process according to the present disclosure may also include total or partial drying of the keratin fibers with a hairdryer.

According to one embodiment, the process for protecting the color of the keratin fibers may comprise a step of heating the composition comprising the at least one unsaturated fatty substance of formula (I), which may then be applied directly to the keratin fibers. The temperature may be, for example, less than or equal to 70° C.

According to another embodiment, the process for protecting the color of the keratin fibers may comprise a step of heating the keratin fibers after application of the composition comprising the at least one unsaturated fatty substance of formula (I).

The heating of the keratin fibers may be performed, for example, using an iron, a liquid water/steam mixture, and/or by means of a heating hood.

The heating iron that may be used in accordance with the present disclosure may be a heating iron conventionally used in the field of haircare, for example, crimping irons and smoothing irons. For example, irons that are useful for implementation of the process of the present disclosure may include, but are not limited to, flat and round irons, such as those described in U.S. Pat. Nos. 4,103,145, 4,308,878, 5,983,903, 5,957,140, and 5,494,058. The iron may be applied by successive separate touches of a few seconds, or by gradually moving or sliding it along the locks. In at least one embodiment, there may be a pause between the application of the color-protecting composition and the application of the heating iron to the keratin fibers. The pause can range from 30 seconds to 60 minutes, for example, from 1 to 30 minutes. The temperature can range, for example, from 60° C. to 120° C.

The liquid water/steam mixture that is useful in accordance with the present disclosure can have a temperature of at least 35° C., for example, greater than or equal to 40° C., or ranging from 40° C. to 75° C.

The liquid water/steam mixture can be in the form of a mist. The mixture may also comprise at least one other gas such as oxygen and nitrogen, mixtures of gases such as air, and other vaporizable compounds.

According to one embodiment, the liquid water/steam mixture is placed in contact with the fiber for a time ranging from 1 second to 1 hour, for example, from 5 minutes to 15 minutes. The application of the mixture may be repeated several times on the same fiber, each operation being performed for a time as indicated above. According to another embodiment, the composition containing the at least one compound of formula (I) is first applied to the hair and these locks thus impregnated are then subjected to the action of the liquid water/steam mixture under the conditions mentioned above, and the locks thus treated are then cooled, for example, by sending over or through them a stream of air chosen from cold air and ambient temperature air.

The liquid water/steam mixture used according to the present disclosure may be produced using any apparatus known in the art and intended for this purpose. In at least one embodiment, an apparatus comprising at least one steam generator directly connected to a hood that diffuses the liquid water/steam mixture onto the keratin fibers, for example, human hair, is used. A non-limiting example of an apparatus of this type is that sold under the name Micromist® by the company Takara Belmont.

Further disclosed herein is a dyeing process comprising applying to human keratin fibers such as the hair, a direct or oxidation dye composition (A) for a time that is sufficient to develop the color, and applying a composition (B) containing, in a cosmetically acceptable medium, at least one unsaturated fatty substance of formula (I) as defined previously, wherein composition (B) may be applied before or after application of composition (A).

The application of composition (A) may be followed by rinsing and/or drying of the keratin fibers.

The application of the composition (B) may be followed by rinsing and/or drying of the keratin fibers. Composition (B) may be preheated under the same conditions defined above. The application of composition (B) may be followed by heating of the keratin fibers under the same conditions defined above.

In at least one embodiment, composition (B) may be applied after applying the direct or oxidation dye composition (A). Composition (B), comprising the at least one unsaturated fatty substance of formula (I) may be applied immediately after dyeing, or after a delay. As used herein, the term “after a delay” means an application that takes place from a few hours up to several days (from 1 to 15 days) after dyeing. According to one embodiment, composition (B) may be applied immediately after dyeing the keratin fibers; and the application of the composition may be repeated between two colorations.

The nature and concentration of the dyes present in the dye composition (A) is not critical and may be chosen according to the general knowledge available in the art.

In the case of lightening direct dyeing operations, the dye compositions (A) result from the mixing, at the time of use, a dye composition (A₁) containing at least one direct dye and a composition (A₂) containing at least one oxidizing agent.

In the case of oxidation dyeing, the dye compositions (A) result from the mixing, at the time of use, a dye composition (A₃) containing at least one oxidation base and optionally at least one coupler and/or a direct dye, and a composition (A₄) containing at least one oxidizing agent.

The direct dyes may be chosen from compounds that absorb light radiation in the visible range (400-750 nm). They may be further chosen from nonionic, anionic, and cationic dyes.

Non-limiting examples of direct dyes include nitrobenzene dyes, and azo dyes, anthraquinone dyes, naphthoquinone dyes, benzoquinone dyes, phenothiazine dyes, indigoid dyes, xanthene dyes, phenanthridine dyes, phthalocyanin dyes, triarylmethane-based dyes, and mixtures thereof.

Suitable nitrobenzene dyes include red and orange compounds, such as 1-hydroxy-3-nitro-4-N-(γ-hydroxypropyl)aminobenzene, N-(β-hydroxyethyl)amino-3-nitro-4-aminobenzene, 1-amino-3-methyl-4-N-(β-hydroxyethyl)amino-6-nitrobenzene, 1-hydroxy-3-nitro-4-N-(β-hydroxyethyl)aminobenzene, 1,4-diamino-2-nitrobenzene, 1-amino-2-nitro-4-methylaminobenzene, N-(β-hydroxyethyl)-2-nitro-para-phenylenediamine, 1-amino-2-nitro-4-(β-hydroxyethyl)amino-5-chlorobenzene, 2-nitro-4-aminodiphenylamine, 1-amino-3-nitro-6-hydroxybenzene, 1-(β-aminoethyl)amino-2-nitro-4-(β-hydroxyethyloxy)benzene, 1-(β,γ-dihydroxypropyl)oxy-3-nitro-4-(β-hydroxyethyl)aminobenzene, 1-hydroxy-3-nitro-4-aminobenzene, 1-hydroxy-2-amino-4,6-dinitrobenzene, 1-methoxy-3-nitro-4-(β-hydroxyethyl)aminobenzene, 2-nitro-4′-hydroxydiphenylamine, 1-amino-2-nitro-4-hydroxy-5-methylbenzene, and mixtures thereof.

The nitrobenzene direct dyes may also include yellow and green-yellow dyes, for instance 1-β-hydroxyethyloxy-3-methylamino-4-nitrobenzene, 1-methylamino-2-nitro-5-(β,γ-dihydroxypropyl)oxybenzene, 1-(β-hydroxyethyl)amino-2-methoxy-4-nitrobenzene, 1-(β-aminoethyl)amino-2-nitro-5-methoxybenzene, 1,3-bis(β-hydroxyethyl)amino-4-nitro-6-chlorobenzene, 1-amino-2-nitro-6-methylbenzene, 1-(β-hydroxyethyl)amino-2-hydroxy-4-nitrobenzene, N-(β-hydroxyethyl)-2-nitro-4-trifluoromethylaniline, 4-(β-hydroxyethyl)amino-3-nitrobenzenesulfonic acid, 4-ethylamino-3-nitrobenzoic acid, 4-(β-hydroxyethyl)amino-3-nitrochlorobenzene, 4-(β-hydroxyethyl)amino-3-nitromethylbenzene, 4-(β,γ-dihydroxypropyl)amino-3-nitrotrifluoromethylbenzene, 1-(β-ureidoethyl)amino-4-nitrobenzene, 1,3-diamino-4-nitrobenzene, 1-hydroxy-2-amino-5-nitrobenzene, 1-amino-2-[tris(hydroxymethyl)methyl]amino-5-nitrobenzene, 1-(β-hydroxyethyl)amino-2-nitrobenzene, and 4-(β-hydroxyethyl)amino-3-nitrobenzamide.

Blue or violet nitrobenzene dyes may also be used, for instance, 1-(β-hydroxyethyl)amino-4-N,N-bis(β-hydroxyethyl)amino-2-nitrobenzene, 1-(γ-hydroxypropyl)amino-4-N,N-bis(β-hydroxyethyl)amino-2-nitrobenzene, 1-(β-hydroxyethyl)amino-4-(N-methyl, N-β-hydroxyethyl)amino-2-nitrobenzene, 1-(β-hydroxyethyl)amino-4-(N-ethyl, N-β-hydroxyethyl)amino-2-nitrobenzene, 1-(β,γ-dihydroxypropyl)amino-4-(N-ethyl, N-β-hydroxyethyl)amino-2-nitrobenzene, and the 2-nitro-para-phenylenediamines of the following formula: 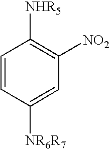
wherein:

R₆ is chosen from C₁-C₄ alkyl radicals, β-hydroxyethyl radicals, β-hydroxypropyl radicals, and γ-hydroxypropyl radicals;

R₅ and R₇, which may be identical or different, are chosen from β-hydroxyethyl radicals, β-hydroxypropyl radicals, γ-hydroxypropyl radicals, and β,γ-dihydroxypropyl radicals, wherein at least one of the radicals R₆, R₇, or R₅ is a γ-hydroxypropyl radical and R₆ and R₇ are not simultaneously β-hydroxyethyl radicals when R₅ is a γ-hydroxypropyl radical, such as those described in French patent FR 2 692 572.

As described herein, azo dyes are compounds comprising in their structure at least one —N═N— sequence not included in a ring; methine dyes are compounds comprising in their structure at least one —C═C— sequence not included in a ring; and azomethine dyes are compounds comprising in their structure at least one —C═N— sequence not included in a ring.

The triarylmethane-based dyes comprise in their structure at least one sequence of formula: 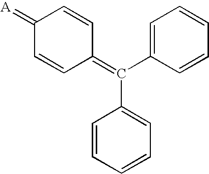
wherein A is chosen from oxygen and nitrogen.

The xanthene dyes comprise in their structure at least one sequence of formula:

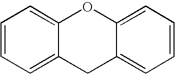

The phenanthridine dyes comprise in their structure at least one sequence of formula:

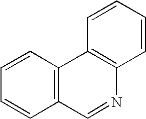

The phthalocyanin dyes comprise in their structure at least one sequence of formula:

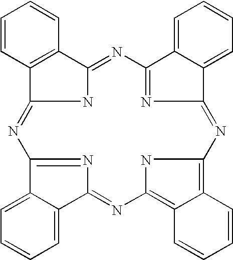

The phenothiazine dyes comprise in their structure at least one sequence below:

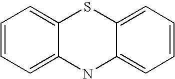

The direct dyes may also be chosen, by non-limiting example, from basic dyes such as those listed in the Color Index, 3rd edition, under the names Basic Brown 16, Basic Brown 17, Basic Yellow 57, Basic Red 76, Basic Violet 10, Basic Blue 26, and Basic Blue 99; the acidic direct dyes listed in the Color Index, 3rd edition, under the names Acid Orange 7, Acid Orange 24, Acid Yellow 36, Acid Red 33, Acid Red 184, Acid Black 2, Acid Violet 43, and Acid Blue 62; and cationic direct dyes such as those described in International Patent Application Publication Nos. WO 95/01772 and WO 95/15144 and European Patent Application No. 0 714 954, which are incorporated herein by reference in their entireties, such as Basic Red 51, Basic Orange 31, and Basic Yellow 87.

When present, the at least one direct dye may be present in the composition in an amount ranging from 0.0005% to 12% by weight relative to the total weight of the composition, for example, from 0.005% to 6% by weight relative to the total weight of the composition.

The oxidation bases may be chosen from the oxidation bases conventionally used in oxidation dyeing, such as, by way of non-limiting example, para-phenylenediamines, bis(phenyl)alkylenediamines, para-aminophenols, ortho-aminophenols, and heterocyclic bases.

Examples of suitable para-phenylenediamines include, but are not limited to, para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, N,N-diethyl-4-amino-3-methylaniline, N,N-bis(β-hydroxyethyl)-para-phenylenediamine, 4-N,N-bis(β-hydroxyethyl)amino-2-methylaniline, 4-N,N-bis(β-hydroxy-ethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetyl-aminoethyloxy-para-phenylenediamine and N-(β-methoxyethyl)-para-phenylenediamine, and the acid addition salts thereof.

According to one embodiment, the para-phenylenediamines may be chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts thereof.

Suitable bis(phenyl)alkylenediamines include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and the acid addition salts thereof.

Non-limiting examples of para-aminophenols include para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and the acid addition salts thereof.

Examples of ortho-aminophenols include, but are not limited to 2-aminophenol, 2-amino-5-methylphenol, 2-amino-6-methylphenol, 5-acetamido-2-aminophenol, and the acid addition salts thereof.

Suitable heterocyclic bases include, for example, pyridine derivatives, pyrimidine derivatives, and pyrazole derivatives.

Non-limiting examples of pyridine derivatives include the compounds described, for example, in British Patent Nos. 1 026 978 and 1 153 196, such as 2,5-di-aminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and the acid addition salts thereof.

Examples of pyrimidine derivatives include, but are not limited to, the compounds described in German Patent No. 2 359 399; Japanese Patent Application No. 88-169 571; Japanese Patent No. 05-163 124; European Patent Application No. 0 770 375, and International Patent Application Publication No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in French Patent Application No. 2 750 048, for example, pyrazolo[1,5-a]pyrimidine-3,7-diamine; 2,5-dimethylpyrazolo[1,5-a]-pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine, 3-amino-5-methyl-7-imidazolylpropylaminopyrazolo[1,5-a]-pyrimidine, the acid addition salts thereof, and the tautomeric forms thereof, when a tautomeric equilibrium exists.

Suitable pyrazole derivatives include, for example, the compounds described in German Patent Nos. 3 843 892, 4 133 957, and 195 43 988, International Patent Application Publication Nos. WO 94/08969 and WO 94/08970, and French Patent No. 2 733 749, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3, 4,5-triaminopyrazole, 3,5-diamino-1-methyl-4-methylaminopyrazole and 3,5-diamino-4-(β-hydroxyethyl)amino-1-methylpyrazole, and the acid addition salts thereof.

When present, the at least one oxidation base may be present in the composition in an amount ranging from 0.0005% to 12% by weight relative to the total weight of the dye composition, for example, from 0.005% to 6% by weight relative to the total weight of the dye composition.

The oxidation dye compositions in accordance with the present disclosure may also comprise at least one coupler and/or at least one direct dye, for example, to modify the shades or to enrich them with tints.

The at least one coupler that can be used in the oxidation dye compositions according to the present disclosure may be chosen from the couplers conventionally used in oxidation dyeing, for example, meta-phenylenediamines, meta-aminophenols, meta-diphenols, naphthols, and heterocyclic couplers, for instance, indole derivatives, indoline derivatives, pyridine derivatives, indazole derivatives, pyrazolo[1,5-b]-1,2,4-triazole derivatives, pyrazolo[3,2-c]-1,2,4-triazole derivatives, benzimidazole derivatives, benzothiazole derivatives, benzoxazole derivatives, 1,3-benzodioxole derivatives, pyrazolones, and the acid addition salts thereof.

According to one embodiment, the at least one coupler may be chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 6-hydroxybenzomorpholine, 3,5-diamino-2,6-dimethoxypyridine, 1-N(β-hydroxyethyl)amino-3,4-methylenedioxybenzene, 2,6-bis(β-hydroxyethyleneamino)toluene, 2,6-dihydroxy-4-methylpyridine, 1H-3-methylpyrazol-5-one, 1-phenyl-3-methylpyrazol-5-one, and the acid addition salts thereof.

When present, the at least one coupler may be present in the composition in an amount ranging from 0.0001% to 10% by weight relative to the total weight of the dye composition, for example, from 0.005% to 5% by weight relative to the total weight of the dye composition.

The dye composition in accordance with the present disclosure may also comprise at least one adjuvant chosen from various adjuvants conventionally used in hair dye compositions, such as anionic, cationic, nonionic, amphoteric, and zwitterionic surfactants and mixtures thereof; anionic, cationic, nonionic, amphoteric, and zwitterionic polymers and mixtures thereof; mineral and organic thickeners; antioxidants; penetrants; sequestrants; fragrances; buffers; dispersants; conditioning agents, for instance, silicones; film-forming agents; preserving agents; and opacifiers.

It is to be understood that a person skilled in the art will take care to select the at least one optional additional compound such that the beneficial properties intrinsically associated with the dye composition in accordance with the present disclosure are not, or are not substantially, adversely affected by the envisaged addition.

The dye composition according to the present disclosure may be in various forms, such as liquids, creams, and gels, or in any other form that is suitable for dyeing keratin fibers such as human hair.

The nature of the at least one oxidizing agent used in the lightening direct dyeing operation (direct dyeing with an oxidizing agent) or in the oxidation dyeing operation is not critical and may be chosen in accordance with general knowledge available in the art.

In at least one embodiment, the at least one oxidizing agent may be chosen from hydrogen peroxide, urea peroxide, alkali metal bromates, ferricyanides, and persalts such as perborates and persulfates. Redox enzymes such as laccases, peroxidases, and two-electron oxidoreductases (such as uricase) may also be used, where appropriate in the presence of the respective donor or cofactor thereof.

According to one embodiment, the process of the present disclosure may be used on hair that has been sensitized by hair treatments other than those mentioned previously in the present disclosure.

Also disclosed herein is also a multi-component dyeing kit comprising at least one first component comprising a direct dye composition (A) and at least one second component comprising a composition (B) containing, in a cosmetically acceptable medium, at least one unsaturated fatty substance of formula (I) as defined herein.

Further disclosed herein is a multi-component dyeing kit comprising at least one first component comprising a composition (A₁) comprising at least one direct dye, at least one second component comprising a composition (A₂) comprising at least one oxidizing agent, and at least one third component comprising a composition (B) comprising, in a cosmetically acceptable medium, at least one unsaturated fatty substance of formula (I) as defined herein.

Still further disclosed herein is a multi-component dyeing kit comprising at least one first component comprising a composition (A₃) comprising at least one oxidation base and optionally at least one coupler and/or at least one direct dye, at least one second component comprising a composition (A₄) comprising at least one oxidizing agent, and a third component comprising a composition (B) comprising, in a cosmetically acceptable medium, at least one unsaturated fatty substance of formula (I) as defined herein.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

Dyeing Step:

At the time of use, an oxidation dye composition for a support, Recital®, containing 6×10⁻⁴ mol % of p-phenylenediamine and 6×10⁻⁴ mol % of 4-amino-2-hydroxytoluene, was mixed weight-for-weight with aqueous hydrogen peroxide solution (professional L'Oréal 20-volumes 6% aqueous hydrogen peroxide solution).

The mixture was then applied to locks of permanent-waved hair containing 90% white hairs, at a rate of 10 g of dye mixture/g of lock. The leave-on time was 15 minutes on each side of the lock. The locks were then rinsed with water, and then washed with DOP Camomile shampoo and dried.

Protective Treatment Steps:

Various pure fatty substances were then applied to a dyed lock, at a rate of 2 grams per gram of hair:

| Applied treatment | Lock |
|---|---|
| Pure oleic acid sold under the reference 01383 by Sigma Aldrich | A |
| Pure linoleic acid sold under the reference L1012 by Sigma Aldrich | B |
| Sorbitan oleate | C |

Next, the treated locks were then left to stand for 30 minutes at 45° C.

The treated locks were then washed twice with DOP Camomile® shampoo so as to remove the residual surface oil.

Untreated locks (references) also underwent these two shampoo washes. The locks were then dried under a hood for 10 minutes at 60° C.

Wash-Fastness Steps:

Washing

A shampoo-fastness test was performed on the above locks with a DOP Camomile® shampoo. 10 successive shampoo washes were performed, with intermediate drying.

Evaluation of the Color Protection

The degradation of the color after washing the treated and untreated locks was evaluated visually relative to unwashed dyed locks.

These evaluations were accompanied by spectrocolorimetric monitoring. Measurements were taken using a Minolta CM2022 spectrocolorimeter, for up to 10 washes.

The degradation caused by the washing is expressed as ΔE:
ΔE(x shampoo washes−0 shampoo washes)=√(ΔL*²+Δa*²+Δb*²)

The protection is then expressed as a difference in ΔE between the treated and untreated locks. (Positive difference=gain in color protection, negative difference=loss in protection, significant difference with a gain ΔE=2).

Results:

After 10 shampoo washes, substantial degradation of the coloration of the untreated dyed locks was observed (loss of ΔE=12.98).

It was observed, surprisingly, that after 10 shampoo washes, only the locks A, B, and C that had been treated with fatty substances of the present disclosure provide a significant color protection compared with the untreated locks.

These results were confirmed by the calorimetric measurements, which indicate a significant gain in ΔE relative to the untreated lock as shown in the table below.

### Results of Color Degradation after Fastness Test of 10 Washes (DOP Camomile® Shampoo)

| | ΔE relative to the | Significant protection relative to the |
|---|---|---|
| Lock | unwashed locks | untreated lock |
| A | 10.31 | Yes |
| B | 10.11 | Yes |
| C | 7.19 | Yes |

What is claimed is:

1. A process for protecting the color of artificially dyed keratin fibers with respect to washing, the process comprising applying to the fibers, before or after dyeing, at least one composition comprising, in a cosmetically acceptable medium, at least one unsaturated fatty substance of formula (I): 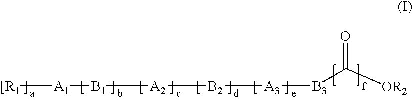
wherein:
A₁, A₂, and A₃, which may be identical or different, are chosen from linear and cyclic, monovalent and divalent hydrocarbon-based radicals comprising at least one unsaturation;
B₁, B₂, and B₃, which may be identical or different, are chosen from CₙH₂ₙ radicals in which n is an integer less than 20;
R₁ is chosen from linear and branched C₁-C₁₂ alkyl radicals;
R₂ is chosen from hydrogen, alkali metals M, and sorbitan groups of formula: 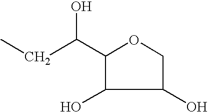
a, b, c, d, e, and f, which may be identical or different, are equal to 0 or 1.

2. The process of claim 1, wherein the at least one unsaturated fatty substance of formula (I) is chosen from the compounds of formula (II): 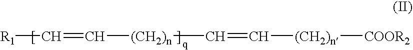
wherein:
R₁ is chosen from linear and branched C₁-C₁₂ alkyl radicals;
R₂ is chosen from hydrogen, alkali metals M, and sorbitan groups of formula: 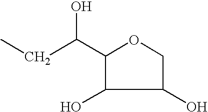
n and n′, which may be identical or different, are integers ranging from 1 to 10; and
q is equal to 0, 1, or 2.

3. The process of claim 1, wherein the at least one unsaturated fatty substance of formula (I) is chosen from:
lauroleic acid;
myristoleic acid;
palmitoleic acid;
oleic acid;
linoleic acid;
linolenic acid; and
sorbitan oleate.

4. The process of claim 3, wherein the at least one unsaturated fatty substance of formula (I) is chosen from oleic acid, sorbitan oleate, and linoleic acid.

5. The process of claim 1, wherein the composition is applied to the fibers after dyeing.

6. The process of claim 1, wherein the cosmetically acceptable medium is chosen from water and mixtures of water and at least one cosmetically acceptable organic solvent.

7. The process of claim 6, wherein the at least one organic solvent is chosen from C₁-C₄ lower alkanols; polyols, and polyol ethers.

8. The process of claim 7, wherein the at least one organic solvent is present in the composition in an amount ranging from 1% to 40% by weight relative to the total weight of the composition

9. The process of claim 8, wherein the at least on organic solvent is present in the composition in an amount ranging from 3% to 30% by weight relative to the total weight of the composition.

10. The process of claim 1, wherein the at least one unsaturated fatty substance of formula (I) is present in the composition in an amount ranging from 1% to 100% by weight relative to the total weight of the composition.

11. The process of claim 1, wherein the at least one unsaturated fatty substance of formula (I) is present in the composition in an amount ranging from 5% to 90% by weight relative to the total weight of the composition.

12. The process of claim 10, wherein the composition comprises 100% by weight of the at least one unsaturated fatty substance of formula (I).

13. The process of claim 1, wherein the composition further comprises at least one additive chosen from anionic, cationic, nonionic, amphoteric, and zwitterionic surfactants; anionic, cationic, nonionic, amphoteric, and zwitterionic polymers; mineral and organic thickeners; penetrants; sequestrants; fragrances; buffers; dispersants; conditioning agents; film-forming agents; ceramides; preserving agents; and opacifiers.

14. The process of claim 1, wherein the composition further comprises at least one agent for protecting against the effects of atmospheric agents.

15. The process of 14, wherein the at least one agent for protecting against the effects of atmospheric agents is chosen from organic UV-screening agents, free-radical scavengers, and antioxidants.

16. The process of claim 1, wherein the composition further comprises at least one aromatic alcohol and at least one aromatic carboxylic acid.

17. The process of claim 1, wherein the composition further comprises at least one conditioning agent.

18. The process of claim 17, wherein the at least one conditioning agent is present in the composition in an amount ranging from 0.001% to 20% by weight relative to the total weight of the composition.

19. The process of claim 18, wherein the at least one conditioning agent is present in the composition in an amount ranging from 0.1% to 3% by weight relative to the total weight of the composition.

20. The process of claim 1, wherein the composition is in a form chosen from aqueous lotions, aqueous-alcoholic lotions, oils, gels, milks, creams, emulsions, and mousses.

21. The process of claim 1, wherein the composition is packaged in a container chosen from vaporizers, pump-dispenser bottles, and aerosol containers.

22. The process of claim 1, wherein the pH of the composition ranges from 1 to 11.

23. The process of claim 22, wherein the pH of the composition ranges from 2 to 6.

24. The process claim 1, further comprising rinsing and/or washing with shampoo before and/or after applying the composition to the keratin fibers.

25. The process of claim 1, further comprising total or partial drying of the keratin fibers with a hairdryer.

26. The process of claim 1, further comprising heating the composition before applying it to the keratin fibers.

27. The process of claim 1, further comprising heating the keratin fibers after application of the composition.

28. The process of claim 27, wherein the heating of the keratin fibers is performed using an iron, a liquid water/steam mixture, and/or a heating hood.

29. A process for dyeing keratin fibers comprising applying to the fibers at least one direct or oxidation dye composition (A) for a time that is sufficient to develop the color, and applying at least one composition (B) containing, in a cosmetically acceptable medium, at least one unsaturated fatty substance of formula (I): 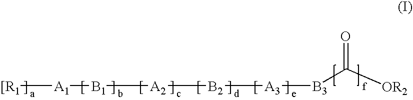
wherein:
A₁, A₂, and A₃, which may be identical or different, are chosen from linear and cyclic, monovalent and divalent hydrocarbon-based radicals comprising at least one unsaturation;
B₁, B₂, and B₃, which may be identical or different, are chosen from CₙH₂ₙ radicals in which n is an integer less than 20;
R₁ is chosen from linear and branched C₁-C₁₂ alkyl radicals;
R₂ is chosen from hydrogen, alkali metals M, and sorbitan groups of formula: 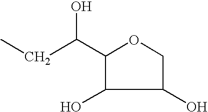
a, b, c, d, e, and f, which may be identical or different, are equal to 0 or 1;
wherein composition (B) is applied to the fibers before or after the application of composition (A).

30. The process of claim 29, wherein the application of composition (A) is followed by rinsing and/or drying of the keratin fibers.

31. The process of claim 29, wherein the application of composition (B) is followed by rinsing and/or drying of the keratin fibers and/or by heating of the keratin fibers.

32. The process of claim 29, wherein composition (B) is preheated.

33. The process of claim 29, wherein composition (B) is applied after applying the direct or oxidation dye composition (A), either immediately or after a delay, and the application of composition (B) is optionally repeated between two colorations.

34. The process of claim 29, wherein the dye composition (A) results from the mixing, at the time of use, of a dye composition (A₁) comprising at least one direct dye, and a composition (A₂) comprising at least one oxidizing agent.

35. The process of claim 29, wherein the dye composition (A) results from the mixing, at the time of use, of a dye composition (A₃) comprising at least one oxidation base and optionally at least one coupler and/or at least one direct dye, and a composition (A₄) comprising at least one oxidizing agent.

36. A multi-component dyeing kit comprising at least one first component comprising a direct or oxidation dye composition (A) and at least one second component comprising a composition (B) comprising, in a cosmetically acceptable medium, at least one unsaturated fatty substance of formula (I): 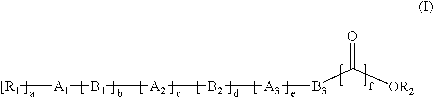
wherein:
A₁, A₂, and A₃, which may be identical or different, are chosen from linear and cyclic, monovalent and divalent hydrocarbon-based radicals comprising at least one unsaturation;
B₁, B₂, and B₃, which may be identical or different, are chosen from CₙH₂ₙ radicals in which n is an integer less than 20;
R₁ is chosen from linear and branched C₁-C₁₂ alkyl radicals;
R₂ is chosen from hydrogen, alkali metals M, and sorbitan groups of formula: 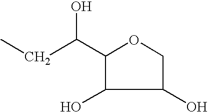
a, b, c, d, e, and f, which may be identical or different, are equal to 0 or 1.

37. A multi-component dyeing kit comprising at least one first component comprising a composition (A₁) comprising at least one direct dye, at least one second component comprising a composition (A₂) comprising at least one oxidizing agent, and at least one third component comprising a composition (B) comprising, in a cosmetically acceptable medium, at least one unsaturated fatty substance of formula (I): 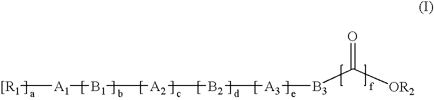
wherein:
A₁, A₂, and A₃, which may be identical or different, are chosen from linear and cyclic, monovalent and divalent hydrocarbon-based radicals comprising at least one unsaturation;
B₁, B₂, and B₃, which may be identical or different, are chosen from CₙH₂ₙ radicals in which n is an integer less than 20;
R₁ is chosen from linear and branched C₁-C₁₂ alkyl radicals;
R₂ is chosen from hydrogen, alkali metals M, and sorbitan groups of formula: 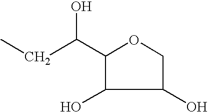
a, b, c, d, e, and f, which may be identical or different, are equal to 0 or 1.

38. A multi-component dyeing kit comprising at least one first component comprising a composition (A₃) comprising at least one oxidation base and optionally at least one coupler and/or at least one direct dye, at least one second component comprising a composition (A₄) comprising at least one oxidizing agent, and at least one third component comprising a composition (B) comprising, in a cosmetically acceptable medium, at least one unsaturated fatty substance of formula (I): 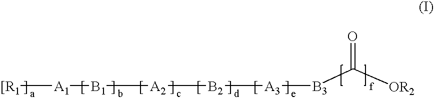
wherein:
A₁, A₂, and A₃, which may be identical or different, are chosen from linear and cyclic, monovalent and divalent hydrocarbon-based radicals comprising at least one unsaturation;
B₁, B₂, and B₃, which may be identical or different, are chosen from CₙH₂ₙ radicals in which n is an integer less than 20;
R₁ is chosen from linear and branched C₁-C₁₂ alkyl radicals;
R₂ is chosen from hydrogen, alkali metals M, and sorbitan groups of formula: 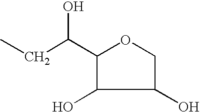
a, b, c, d, e, and f, which may be identical or different, are equal to 0 or 1.
